(19) United States
(12) Patent Application Publication
(10) Pub. No.: US 2008/0274552 A1
(43) Pub. Date: Nov. 6, 2008

(54) Dynamic Information Transfer

(76) Inventors: Brian Guthrie, Inverness (GB); Eng Kim Shuy, Singapore (SG); Gordon George Sansom, Scaniport (GB); David Taylor, Alloa (GB); Keith Torrie, Inverness (GB); Robert Marshall, Canon Bridge (GB); David Howarth, Holm (GB); Emma Day, Dingwall (GB)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/744,707

(22) Filed: May 4, 2007

(51) Int. Cl. G01N 33/487; G01N 31/00

(52) U.S. Cl. 436/8; 422/55; 422/58; 422/61; 422/68.1

(57) ABSTRACT

Disclosed are various preferred embodiments for dynamic transfer of information from a test sensor to an analyte medical test device. Exemplary embodiments include various containers, systems and methods.

### BACKGROUND

Systems for measuring the concentration of a specific analyte or indicator from a sample of whole blood, plasma or interstitial fluid are commonly known and documented. For many individuals who suffer from diabetes, measurement of their blood glucose levels is a necessary part of daily life. Patients are advised by their health care professional to monitor their blood sugar levels regularly each day, typically ranging between two and six tests per day. To do this, measurement systems are commercially available that typically include a meter, disposable test sensors and lancets, such as the OneTouch® Ultra from Lifescan Inc., Milpitas, USA.

Diabetics are often given a blood glucose meter by their healthcare professional (HCP), or they may have decided to purchase one. The process of manufacture of test sensors (also known as test strips) for use with such a meter may be subject to a degree of variability between batches of test strips. In order to correct for this variability, each batch of test strips is assigned a calibration code to define die calibration slope and intercept parameters (“predetermined calibration parameters”) of such batch so as to correlate the predetermined calibration parameters to respective calibration codes recognisable by the meter. The calibration code reduces variability in the different batches of test strips, ensuring that the results obtained using test sensors from different batches will be generally equal and consistent by application of an algorithm that adjusts any difference in the response of the strips to the analyte being measured. Each time a user purchases a new packet of test strips (taken hereto to include packaging of single test strips within such packaging, as will be described herein, and also a container, cartridge or dispenser or other means of housing a plurality of test strips) the batch of test strips will have assigned to it one of a number of different calibration codes. It is possible for the new test strips to have the same calibration code as the previous packet used; however it is likely that it will be different. One example of how calibration parameters are determined and categorized as calibration codes for analyte test strips is shown and described in U.S. Pat. No. 6,780,645, which is incorporated by reference in its entirety herein.

Most meters currently available require the user to read the calibration, code assigned to the new strips and manually enter this code into the meter prior to use. Calibrating the meter each time a new packet of strips is started, or indeed each time the user wishes to perform a test, can be inconvenient due to the number of steps involved and the time consuming process of having to check the calibration code printed on the label of the vial. It is potentially inconvenient for the user to perform this step, particularly if the code required is printed on packaging that could have been discarded or if the user is in a hurry, for example, experiencing a period of hypoglycemia when their thought processes may not be at its optimum. Looking for small print on a label can be problematic for many diabetics as diminished eyesight is often a resultant complication of the disease. Users may forget to enter the calibration code or they may decide not to enter it if they do not understand its significance. Obtaining a result, such as a blood, glucose concentration from a meter and ship system that is not properly calibrated, may be incorrect and potentially harmful to the user. An incorrect result may cause them to take inappropriate action.

For reasons including those described herein, applicants recognize that it is desirable for the measurement system to include automatic calibration and to reduce the number of steps required by the user in order to perform a measurement. As the need to measure analyte concentrations in physiological samples increases due to the growing occurrence of diabetes and the importance of closely managing the disease, applicants recognize that there is increased demand for a measurement system that is all-inclusive, compact, easy to use, fast and includes few user steps.

### BRIEF SUMMARY

In one preferred, embodiment, a container is provided that includes an interior surface, a test strip, outer surface, and first and second pluralities of discrete surface features. The interior surface defines an internal volume. The single test strip is disposed in the internal volume, the single test strip having at least a predetermined calibration code corresponding to predetermined calibration parameters specific to the test strip. The outer surface surrounds the interior surface. The first plurality of discrete surface features are disposed on the outer surface to define a first data line. The second plurality of discrete surface features are disposed on the outer surface in a repeating sequence to define timing intervals for the first data due so that the predetermined calibration code of the single test strip is encoded by the first and second plurality of discrete surface features with the container.

In yet another embodiment, a system is provided that includes a test media container and a medical test device. The test media container that includes an interior surface, a test strip, outer surface, and an outer surface that surrounds a substantial portion of the interior surface. The outer surface has discrete surface features indicative of the predetermined calibration code of the at least one test strip. The test device includes a sample port and a pattern reader. The sample port is configured to receive the test strip container in only one spatial orientation. The pattern reader is configured to recognize at least the calibration code encoded in the discrete surface features upon relative movement between the container and the sample port.

These and other embodiments, features and advantages will become apparent to those skilled in the art when taken with reference to the following more detailed description of the invention in conjunction with the accompanying drawings that are first briefly described.

### BRIEF DESCRIPTION OF THE DRAWINGS

The accompanying drawings, which are incorporated herein and constitute part of this specification, illustrate presently preferred embodiments of the invention, and, together with the general description given above and the detailed description given below, serve to explain features of the invention.

FIG. 1A is a perspective view of an exemplary embodiment of a container according to die present invention;

FIG. 1B is a simplified schematic view of an example meter or test device for use with the container of FIG. 1A;

FIG. 2A is a perspective view of the container 2′ of FIG. 1B;

FIG. 2B is a perspective view of a variation of the container 2′ having nubs or raised surfaces on the outer surface of the container;

FIG. 2C is a perspective view of a variation of the container 2′ having depressions on the outer surface of the container;

FIG. 3 is a close-up perspective view of the meter of FIG. 1B showing the location of calibration optical sensors;

FIG. 4 is a process don diagram outlining the main steps involved in the use of the container and meter of FIGS. 1A, 1B and 3;

FIG. 5 is a close-up top plan view of an example embodiment of a coded region located on the container of FIG. 1A;

FIG. 6 is a table comprising 16 calibration code zones and 64 different permutations of calibration codes;

FIG. 7 is an oscilloscope wave chart of the optical detection of the calibration code of FIG. 5, showing the wave form when a container is withdrawn from a meter relatively quickly;

FIG. 8 is an oscilloscope wave chart of the optical detection of a the calibration code of FIG. 5, showing the wave form when a container is withdrawn from a meter relatively slowly;

FIG. 9 is a process flow diagram outlining the process of interrogating the coded information of FIG. 5 by optical sensors within the meter housing;

FIG. 10a illustrates an exemplary code pattern for use in the transfer of specific information;

FIG. 10b is an alternative example code pattern for use in the transfer or specific information;

FIG. 11 is a cross-section view through strip port hay of the meter of FIG. 1B showing the relative locations of calibration sensors and a strip presence detection sensor according to an embodiment of the present invention;

FIG. 12 is a schematic view of a test sensor with integral lancet for use with the container of FIGS. 1A, 1B, and 3;

FIG. 13 is a flow diagram, outlining the main steps involved in the procedure of loading a new test strip into the meter of FIG. 1B;

FIG. 14 illustrates an exemplary oscilloscope wave chart obtained if a test strip, including integral lancet portion, is absent or incorrectly loaded into strip port connector;

FIG. 15 illustrates an exemplary oscilloscope wave chart of the optical detection of the test strip of FIG. 12, detected once the container of FIG. 1A is withdrawn from the meter.

### DETAILED DESCRIPTION

The following detailed description should be read with reference to the drawings, in which like elements in different drawings are identically numbered. The drawings, which are not necessarily to scale, depict selected embodiments and are not intended, to limit the scope of the invention. The detailed description illustrates by way of example, not by way of limitation, the principles of the invention. This description will clearly enable one skilled in the art to make and use the invention, and describes several embodiments, adaptations, variations, alternatives and uses of the invention, including what is presently believed to be the best mode of carrying out the invention.

As used herein, the terms “about” or “approximately” for any numerical values or ranges indicate a suitable dimensional tolerance that allows the part or collection of components to function for its intended purpose as described herein.

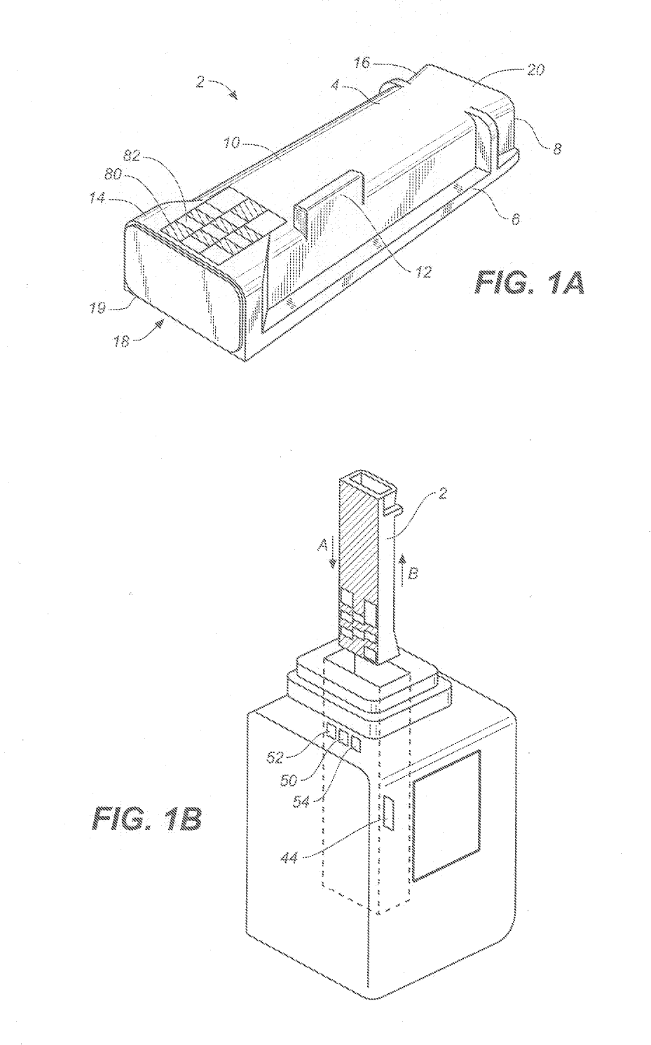

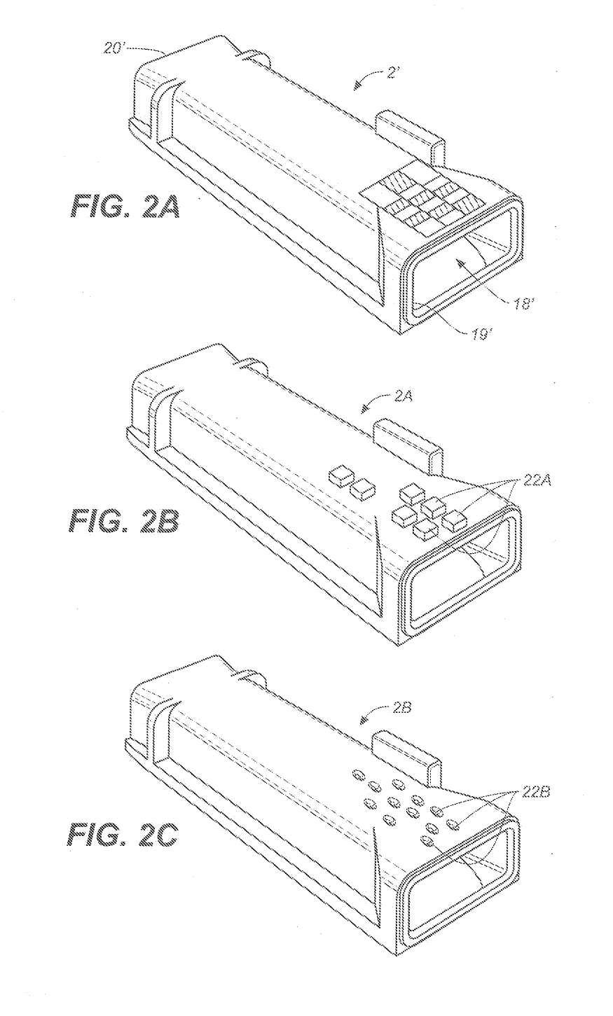

FIG. 1A is a perspective view of an example embodiment of a container 2 including a ton surface 4, a bottom surface 6, a first side 8, a second side 10, a protrusion 12 on first side 8, a proximal end 14, a distal end 16, a main cavity 18 with an opening 19, a secondary cavity 20, a code region 22, dark patches 82 and light patches 80. FIG. 2A illustrates a variation 2′ of the container 2 in FIG. 1A.

Container 2 or 2′ may be molded in a single piece from a rigid material such as high-density polyethylene for example, alternatively including desiccant, available from Airsec, Barcelona, Spain. Referring to FIGS. 1A and 2A, container 2 or 2′ includes a proximal end 14 and a distal end 16 with a main internal cavity 18 extending from proximal end 14, and a secondary cavity 20 extending from distal end 16. Main cavity 18 has an opening 19 at proximal end 14 of container 2 configured to receive, and to securely and removably retain a medical device at least partially therein e.g. a test sensor alternatively including an integrated lancet such as the test sensor described in detail in patent application WO2005/0061700A1 filed on Sep. 19, 2003 by the same applicant, the entire contents of which are included herein by reference. Proximal end 14 may alternatively be covered with a suitable material such as a metallized (e.g., aluminum) foil for example to maintain sterility of the medical device and provide protection from damage and exposure from light and/or moisture. The foil is preferably used to hermetically seal the test strip within the container 2. Secondary cavity 20 extending from distal end 16 may be open, and is intended to receive and safely store the medical device after use.

Each individual container 2 contains a single medical device, i.e. a test strip for the measurement of an analyte, and has assigned to it a calibration code specific to the manufactured batch or lot to ensure the result obtained for every test strip is calibrated, for any minor differences in the manufacturing process. Top surface 4 of container 2 contains a code region 22 comprising of discrete surface features. In one preferred embodiment, the discrete surfaces features include a number of data columns of surface indicia. In particular, the surface indicia includes suitably reflective and non-reflective surfaces providing either a high or a low reflectance capable of being read or recognized by a suitable pattern reader. In one example embodiment, code region 22 comprises dark patches 82 and light patches 80 alternatively configured in 3 columns of coded information as shown in FIG. 1A, and will be described in more detail in relation to FIG. 5. Code region 22 may be located on top surface 4 alternatively close to proximal end 14 to facilitate reading of the coded information by optical sensors housed within a meter as will be described in relation to FIGS. 5 to 9. In another embodiment, shown here in FIGS. 2B and 2C, the discrete surface features can include positive and negative surface features instead of surface indicia.

Container 2 may be used alone or preferably in conjunction with a corresponding meter, such as the example embodiment of a meter shown in FIG. 1B, designed specifically to receive a medical device such as a test strip used for the determination of the concentration of an analyte of interest, such as blood glucose monitoring by patients with diabetes.

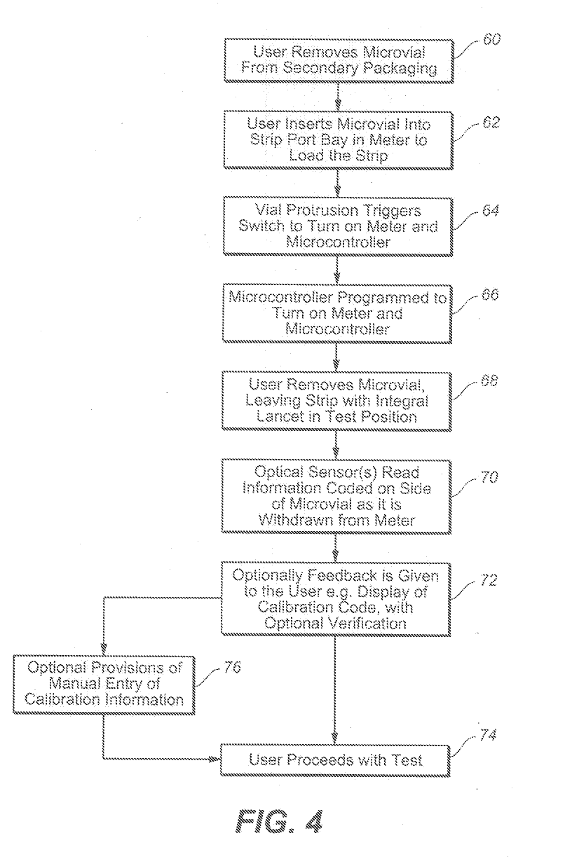

First side 8 of container 2 may include a protrusion 12 that performs two different functions. First protrusion 12 ensures that the user can only insert container 2 into a cooperating meter in one spatial orientation, and secondly, protrusion 12 triggers a switch (item 44 in FIG. 1B) on insertion of container 2 into a receiving cavity or port within a meter that activates the meter to turn on as will be described in relation to FIGS. 1B and 4. Alternative methods of switching may be used in place of mechanical switch 44, such as a reflective photo-interrupter switch for example, activation of which would detect the presence of a container 2 and hence power on the meter.

FIG. 1B illustrates a simplified schematic perspective view of an exemplary meter 30 for use with the container 2 of FIG. 1 including a meter housing 32 with a front side 32a and a back side 32b, a hinged door 34, a display 36, user operable buttons 38, a strip port bay 42 to receive container 2 including a protrusion 12, a clock line optical sensor 50, a first data line optical sensor 54, a second data line optical sensor 52, a mechanical switch 44, an arrow ‘A’ indicating the direction of insertion of container 2 into meter 30 and an arrow ‘B’ indicating the direction of removal of container 2. Meter 30 alternatively includes a hinged door 34 to protect the internal components of meter 30 from damage by sharp objects or exposure to dust particles or moisture for example, and is required to be opened each time a user wishes to test.

In operation, a user first inserts a new container 2 (or 2′) into strip port bay 42 of meter housing 32, in the direction of insertion as indicated by arrow ‘A’. On insertion of container 2 into strip port bay 42, protrusion 12 on first side 8 of container 2 triggers a switch 44, which activates meter 30 to power on out of standby mode.

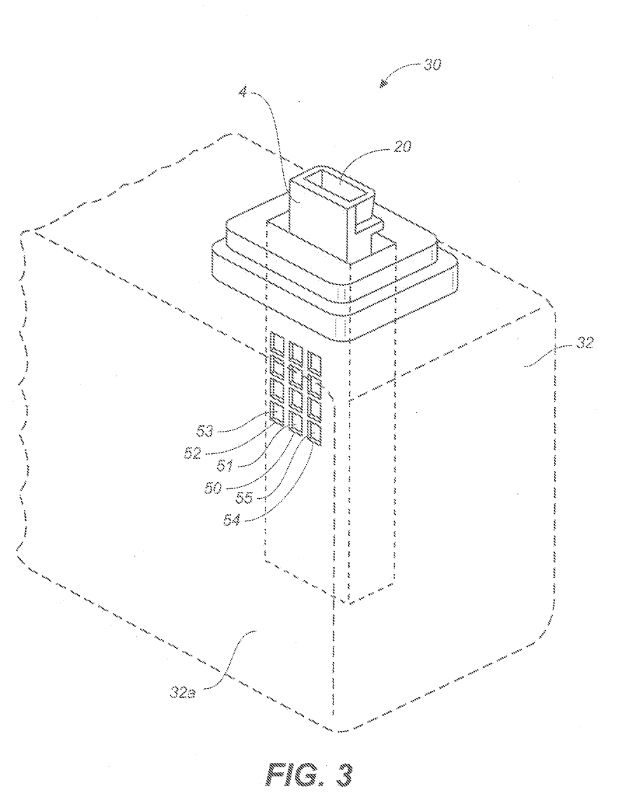

Part of the power-on sequence controlled by a micro-controller (not shown) instructs optical sensors 50, 52 and 54 (shown in detail in FIG. 3) to turn on and interrogate code region 22 on top surface 4 of container 2 as it is withdrawn from strip port bay 42, in a direction indicated by arrow ‘B’. When container 2 is removed from strip port bay 42, a new test strip, alternatively including an integrated lancet, is loaded in the test position and hinged door 34 would be closed and the meter 30 ready for use. Although exemplary meter 30 is shown herein with a hinged door 34, meter 30 may not require a hinged door or may utilize a different, type of cover such as, for example, a snap-fit cover.

User operable buttons 38 located on meter housing 32 provide the user with the ability to operate the meter 30 in accordance with any instructions shown on display 36, and subsequently the measurement result will also be available for viewing on display 36. The calibration procedure may alternatively be visible to the user in the form of a brief display of the code retrieved, followed by an optional request for user verification. Alternatively, the calibration procedure may be completely invisible to the user. A more detailed description of the operation of meter 30 is provided in relation to FIGS. 4 and 9.

Such a meter is intended to be pocket-sized and easy for a patient such as a person with diabetes to regularly test their blood sugar concentration, allowing them to take appropriate action such as medication or diet control in order to maintain a healthy lifestyle.

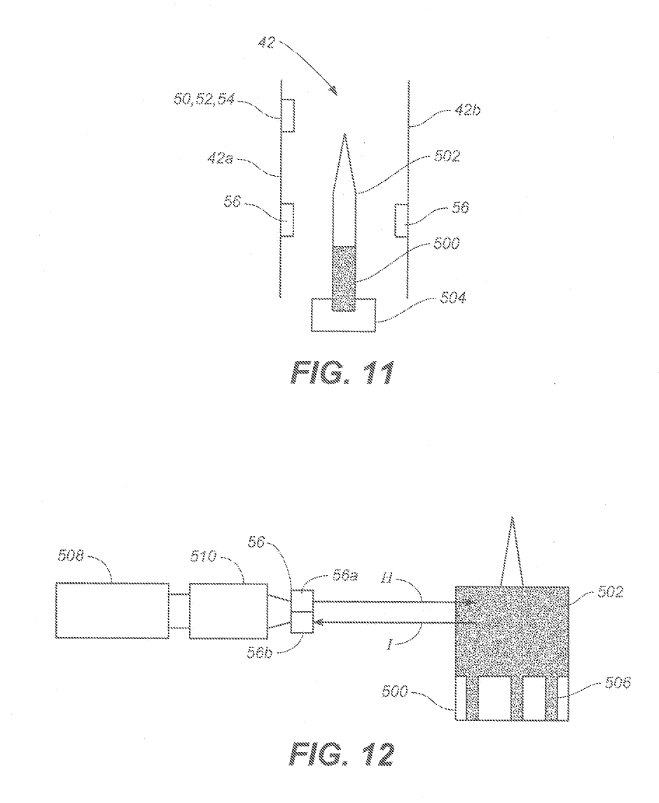

FIG. 3 is a close up perspective view of the meter 30 of FIG. 1B including optical sensors for use with container 2 of FIG. 1, including a meter housing 32 with a front side 32a and a hack side 32b, a strip port bay 42, a top surface 4 of container 2, a clock line sensor 50 adjacent to a corresponding window 51, a first data line sensor 54 adjacent to a corresponding window 55, a second data line sensor 52 adjacent to a corresponding window 53 and a line A-A depicting the cross-section view of FIG. 11.

Optical sensors 50, 52 and 54 may be located on a printed circuit board (PCB) either separately or combined to form a single component, alternatively mounted within a housing of dark opaque material comprising corresponding windows 51, 53 and 55 respectively, Windows 51, 53 and 55 surround the individual sensing elements 50, 52 and 54 thereby preventing unwarned transmission of optical signal there-between. Optical sensors 50, 52 and 54 may include, in one exemplary embodiment, an infrared sending element and receiving element in close proximity, and may be located near to the entrance of strip port bay 42 as shown in FIG. 1B, to ensure reliable detection and reading of code region 22 on container 2 as it is withdrawn in a direction indicated by arrow B. In an alternative embodiment, sensors 50, 52 and 54 may alternatively be isolated from each other by communicating the optical signals by means of clear tube components, thereby creating a light pipe by means of internal reflection for each sensing element. Alternative light sources may include different types of light emitting diodes, such as a laser diode for example.

As described in relation to FIGS. 2 and 4, container 2 triggers switch 44 that activates an internal microprocessor and subsequently powers on optical sensors 50, 52 and 54. As container 2 is removed, from strip port bay 42, clock line sensor 50 reads clock line 84, first data line sensor 54 reads first data line 86 and second data line sensor 52 reads second data line 88 dynamically, interrogating the data bits of code region 22 individually, and the retrieved information is subsequently transferred to the microprocessor for de-coding and transformation into a calibration code to be used in the calculation of the analyte concentration being measured. It is necessary for the calibration information to be transferred to the meter memory prior to a test being performed to ensure that the information is available to be used in the calculation of the final measurement result displayed to the user.

In one exemplary embodiment, code region 22 is read dynamically as container 2 is inserted or withdrawn from strip port bay 42 leaving a new test strip loaded in the test position. In an alternative embodiment, container 2 may alternatively be static with respect to the optical sensors, however this would require one sensor for each bit of information contained within the code, taking up valuable space on the PCB and potentially increasing cost of the measurement system. In a further embodiment, code region 22 may be read on insertion of container 2 into strip port bay 42. This measurement would however take place prior to a test strip being successfully loaded into meter 30. It is one of many advantages herein to ensure a strip is correctly loaded and ready for use prior to code region 22 being read by optical sensors 50, 52 and 54.

Component number GP2S60 is art example of an optical sensor that may be used with the preferred embodiment, available from Sharp Electronics UK Ltd., Uxbridge. Other types of sensors may alternatively be used, such as transmissive optical sensors or an optical switch used in conjunction with the presence or absence of holes in the component being detected for example, as alternatives to the use of reflective optical switches or sensors.

FIG. 4 is a generalized process flow diagram outlining the main steps involved in the use of container 2 and meter 30 of FIGS. 1, 2 and 3. A user first removes a new container 2 from any secondary packaging, step 60, and inserts container 2 info strip port bay 42 in meter 30, step 62. As container 2 is inserted into strip port bay 42, a protrusion 12 located on one side of container 2 triggers a switch 44 that powers on meter 30 and a micro-controller (not shown), step 64. The micro-controller is programmed to turn on optical sensors 50, 52 and 54, step 66. As container 2 is withdrawn (leaving meter 30 loaded with a test strip in the test position ready to perform a test), step 68, optical sensors 50, 52, and 54 interrogate code region 22 located on top surface 4 of container 2, step 70, and output the information retrieved to the micro-controller for interpretation and subsequent use in the analyte concentration calculation. Alternatively, an output in the form of a suitable feedback may be provided to the user, step 72, e.g. in the form of an audible beep or a message briefly displayed on display 36, such that the user may alternatively be requested to verify the calibration information obtained, from optical code region 22. The user may then proceed with the measurement procedure step 74, knowing that meter 30 is calibrated for the particular batch of test strips being used.

Alternatively, the procedure of calibration may be completely invisible to the user, with no display of information or request for confirmation. An option for manual input of information such as calibration code may be included, step 76, allowing the user to proceed with a test knowing that the system is properly calibrated in the unlikely event that there is a fault in the operation of any of optical sensors 30, 52 and 54.

FIG. 5 is a close-up top plan view of an exemplary embodiment of a code region 22 located towards a proximal end 14 of top surface 4 of the exemplary container 2 of FIG. 1. Code region 22 includes dark patches 82, light patches 80, a central clock line 84, a first data line 86 and a second data line 88, a first clock edge 81, a second clock edge 83, a third clock edge 85, a fourth clock edge 87, a fifth clock edge 89, and lengths ‘c’, ‘d’, ‘e’, ‘f’ and ‘g’ of a series of data bits comprising clock line 84.

Code region 22 includes a plurality of dark patches 82 and light patches 80; the light and dark patches (80, 82) forming distinct columns of information beginning at proximal end 14 and partially covering top surface 4 of container 2 in the direction towards distal end 16. The exemplary embodiment of a code region 22 provided in FIG. 5 shows 3 columns or rows of information; a clock line 84 in the center with, a first data line 86 on the side and a second, data line 88 on the other. Dark patches 82 may alternatively include the same material from which container 2 is manufactured e.g. desiccated polyethylene, or may include a suitably dark, non-reflective ink or media. Light patches 80 may be composed of any suitably reflective ink such as silver or Iriodin® pigment for example, deposited onto container 2 using deposition methods such as pad printing, stamping, laser printing or etching during the manufacturing process. A suitable ink will also be one that does not scratch off easily during use and/or storage. Alternatively, a clear coating may be applied for protection of code region 22. In yet a further alternative, a degradable ink can also be utilized for instances where the duration of the ink is about the same as the duration of the shelf-life of a test strip so that the calibration code cannot be read at about the same time the test strip is expiring or a suitable number of months before or after expiration of the strip. Additionally, surface modifications can be made via a laser to produce the dark/light contrasts required.

Whilst the embodiment of code 22 shown in FIG. 5 shows alternating light 80 and dark patches 82, this is provided as one example only, and it would be apparent to a person skilled in the art that any order, number and configuration of light 80 and/or dark patches 80 can be utilized, and which are intended to be included within the scope of the disclosure. The size and/or pattern of code region 22 may be dependent upon the amount of information to be stored, size limitations of the manufacturing method or the code reading apparatus. Although the exemplary embodiment of a container 2 shown herein is dark in color and having light patches 80 placed thereon to form code region 22, it would be apparent to a person skilled in the art that any color of container can be utilized, and code 22 may include regions of high and low reflectivity, different level of fluorescence or some detectable change in the surface properties of container 2 such as, for example, magnetic property being imparted to the container to correspond to the coding region in the form of magnetic ink or magnetic particles, in an alternative embodiment, surface modifications may include, for example, punching the code into the surface of container 2 using a binary system similar to that described herein. The code may then consist of a pattern of raised regions or depressions, and detection may either take the form of optical or proximity sensors.

Code region 22 includes one clock line 84 and two data lines (first data line 86 and second data line 88). Clock line 84 and data lines 86 and 88 could be placed in any arrangement, for example clock line 84 may be located towards the outside edge of top surface 4 and either first data line 86 or second data line 88 may be positioned in the center. However placing clock line 84 in the center increases the tolerance to any variability in alignment of code region 22 with the location 40 of optical sensors 50, 52 and 54 when container 2 is inserted into strip port bay 42 in meter 30 designed specifically to receive container 2. Each column of coded data has a corresponding optical sensor located within meter housing 32, for example first sensor 54 may read first data line 86, a second sensor 50 may read clock line 84, and a third sensor 52 may read second data line 88 as described in relation to FIG. 3. It would be apparent to a person skilled in the art that an optical code region 22 may include any number of dark and light patches, configured in any orientation and may be interrogated by a single sensor or multiple sensors and is not intended to be restricted to the example provided herein.

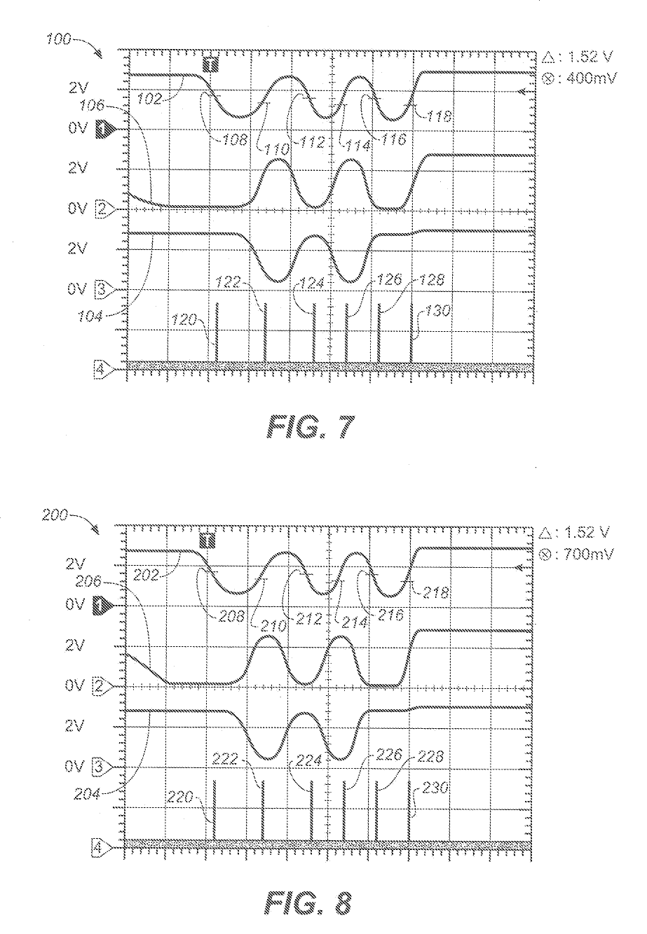

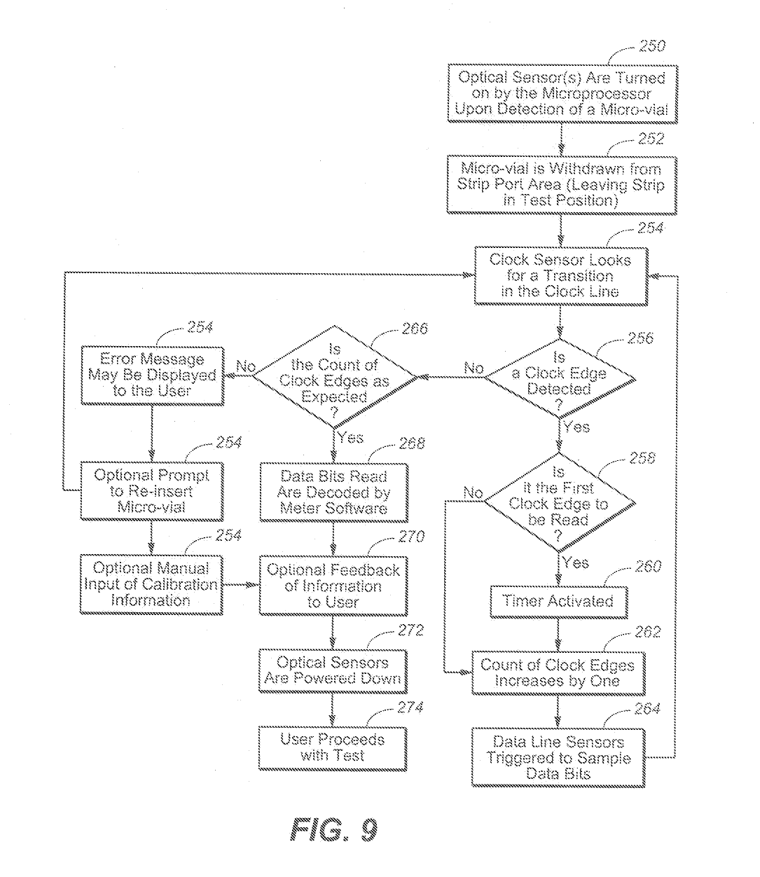

When container 2 is inserted in an associated meter 30, in a direction indicated by arrow A as described in relation to FIGS. 1B and 4, code region 22 is read as container 2 is withdrawn in a direction indicated, by arrow B. Clock line 84 may be used for timing purposes so that first data line sensor 54 reads the coded information in first data line 86, and second data line sensor 52 reads the coded information in second data, line 88 concurrently at the intervals defined by clock line 84. Clock edges 81, 83, 85, 87 and 89 in clock line 84 trigger first and second data line sensors 54, 52, to sample and read first and second data lines 86, 88 respectively, as described in more detail in relation to FIGS. 7 and 8.

The length, dimension of light patches 80 and dark patches 82 may be equal or may alternatively differ to account for variability in the speed at which user pulls container 2 out of strip port bay 42, as indicated by lengths ‘c’, ‘d’, ‘e’, ‘f’ and ‘g’ of data hits in clock line 84. Patches may be larger where the withdrawal, speed is expected to be greatest, and similarly the patches may be smaller in length dimension where the speed of withdrawal is expected to be slower, in one exemplary embodiment, as container 2 is withdrawn in a direction indicated by arrow B, clock line sensor 50 reads clock line 84 and first detects a light patch 80 of length ‘c’ approximately 3.8 mm, followed by a dark patch 82 of length ‘d’ approximately 1.9 mm, followed by a light patch 80 of length ‘e’ approximately 1.9 mm, followed by a dark patch 82 of length ‘f’ approximately 1.95 mm, followed by a final light patch 80 of length ‘g’ approximately 2.0 mm.

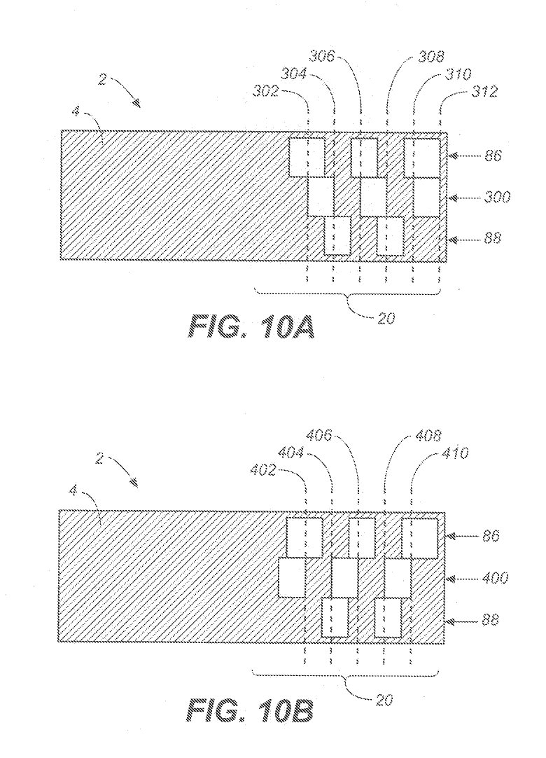

Clock line 84 may alternatively include the same pattern of data bits on every container manufactured, whilst the configuration of light 80 and dark patches 82 in first and second data lines 86, 88 differ according to the batch-specific calibration code. Alternatively, the pattern of clock line 84 may be varied to alter containers 2 intended for different markets, providing benefits to the user such as automatically launching the corresponding meter in the correct language setting for example, showing a welcome or splash screen in the correct language or even providing the user with the country specific customer services telephone number, described in more detail in relation to FIGS. 10a and 10b.

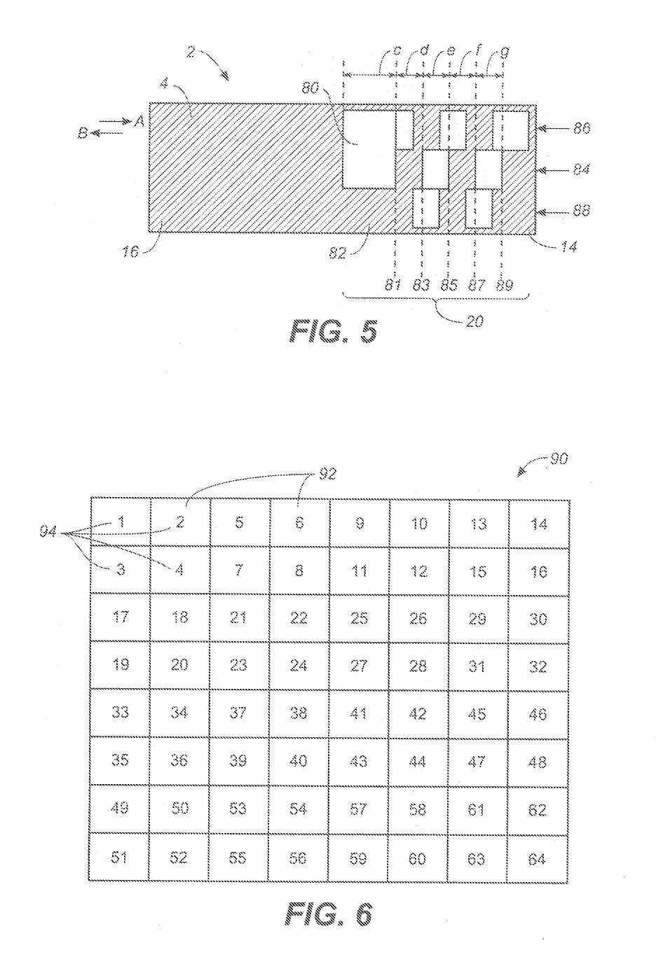

FIG. 6 shows a table 90 comprising 16 calibration code zones 92 and 64 different permutations of calibration codes 94. First and second data lines, 86 and 88 respectively, include data bits coded within light patches 80 and dark patches 82, that together make up information such as a calibration code for example. Data bits may also be used for the detection of errors. The exemplary embodiment of a code region 22 shown in FIG. 5 includes 10 data bits within first 86 and second 88 data lines; 10 data bits could provide 2¹⁰ permutations of information such as a calibration code. Commercially available test strips such as the OneTouch® Ultra brand from Lifescan, Inc. Milpitas, Calif., USA utilize about 49 calibration codes, therefore it is anticipated that approximately 49 calibration codes would be required. With 10 data bits available, numerous possibilities in coding and error checking are available. One such exemplary embodiment may utilize 6 data bits for calibration coding and the remaining 4 data bits for error detection. 2⁶ different calibration codes would be available with 2⁴ data bits remaining for error detection schemes. Another example may be to use 4 data bits to protect the 4 most significant data bits of the calibration code by standard error detection means such as Hamming code for example, which provides 16 calibration code zones, each split into 4 thereby generating 64 different permutations of calibration code as shown in FIG. 6. Errors such as correct operation of the optical sensors and whether or not the expected number of clock edges has been detected would be examples of types of errors detectable within such an error detection scheme. Alternatively, other data can be included with the data lines such as, for example, geographical data, lot number, manufacturing date, expiration date, batch number, manufacturer's name, chemical composition, ingredients, and combinations thereof.

FIG. 7 is an oscilloscope wave chart 100 generated by an optical code of the present invention, such as code region 22 of FIG. 5, showing the change in voltage measured when a container 2 is withdrawn from a meter 30 relatively quickly, including a clock line voltage 102, a first data line voltage 104, a second data line voltage 106, clock edges 108, 110, 112, 114, 116, 118 and sampling pulses 120, 122, 124, 126, 128 and 130.

FIG. 8 is a further oscilloscope wave chart 200 generated by the discrete surface features of the preferred embodiments, such as code region 22 of FIG. 5, showing all the same features of FIG. 7, the difference being the rate of change of voltage as a container 2 is withdrawn from a meter 30 relatively slowly.

As container 2 is withdrawn from strip port bay 42, optical sensors 50, 52 and 54 interrogate the corresponding columns of coded, data. The quantity of light reflected from both the light 80 and dark 82 patches, and received by a cooperating detector is subsequently converted to a voltage that is measured by an analogue to digital converter within the microprocessor (not shown). The voltage detected from a light patch 80 may be in the region of 0.3V, compared to a voltage of approximately 2.8V for a dark patch 82, sufficiently different to be distinguished between by the microprocessor. A transition from a light patch 80 to a dark patch 82 or from a dark patch 82 to a light patch 80 is defined here;a as a clock edge. Detection of a voltage of less than 1.3V for example i.e. interrogation of a light patch 80, would generate a ‘0’ in binary code, and detection of a voltage greater than 1.7V i.e. a dark patch 82, generates a ‘1’ binary code. Data bits in first and second data lines (86 and 88 respectively) of code region 22, such as the example shown in FIG. 5, therefore generate a series of 0's and 1's providing a calibration code in binary format that is transformed into meaningful calibration information by the microprocessor. Optical sensors 50, 52 and 54 may alternatively be calibrated at manufacture to such predefined values, e.g. 1.3V and 1.7V.

FIGS. 7 and 8 snow typical sinusoidal waveforms of the voltage detected as the light 80 and dark 82 patches are interrogated by optical sensors 50, 52 and 54 during the dynamic removal of container 2 from strip port bay 42. Clock line voltage 102 is generated when clock line sensor 50 reads die coded data on clock line 84 (shown in FIG. 5) and each time there is a transition in voltage below a first threshold value e.g. 1.3V or above a second threshold value e.g. 1.7V, a clock edge is detected and first and second sensors (54, 52) are triggered to sample the information within first and second data lines (86, 88) at this point in time, generating first data line voltage 104 and second data line voltage 106. If the voltage detected by first or second sensors (54, 52) is less than a predetermined threshold value, for example 1.5V (i.e. measure of a light patch 80) then a ‘0’ is recorded, and if the voltage detected is above this same threshold value i.e. >1.5V (indicating measure of a dark patch 82) then a ‘1’ is recorded, thereby generating a binary code containing information such as a calibration code that may be transformed and stored for subsequent use by the micro-processor.

Clock line voltages 102 and 202 shown in FIGS. 7 and 8 respectively, show a first clock edge 108, 208 detected when the voltage drops below 1.3V (i.e. transition from a dark 82 to a light patch 80) and triggers sample 120, 220. In the exemplary embodiment shown in FIGS. 7 and 8 a ‘1’ would be read from first data lines 104, 204, and a ‘0’ would be read from second data lines 106, 206. As container 2 continues to be withdrawn, a rise in voltage above 1.7V (i.e. transition from light 80 to a dark patch 82) gives a second clock edge 110, 210 and triggers samples 122, 222, generating a ‘1’ from first data lines 104, 204 and a ‘0’ from second data lines 106, 206 and so on until the end of code region 22 is reached. A binary code of 1,0,0,1,1,0,0,1,1,0 would therefore be determined by the microprocessor for the example shown in FIGS. 7 and 8, and subsequently transformed into a useable calibration code and stored in the meter memory for use in the final calculation of the concentration of an analyte of interest e.g. blood glucose.

A further advantage of the preferred embodiments is to include at least one transition detection, i.e. transition from a dark 82 to a light patch 80 or vice versa, in each data or clock line. Detection of a transition line therefore ensures that the optical sensors are indeed operating correctly, and provides a useful means of self-checking the optical sensors each time they are powered on by insertion of a container 2, without the need for a separate software routine to provide this function.

If optical sensors 50, 52 and 54 experience some interference by natural sunlight, in geographic regions with very bright ambient lighting, for example, this may produce a much longer voltage pulse than, the typical pulse duration expected from data bits in code region 22, therefore the microprocessor can be programmed to ignore such longer pulses. Similarly, if there is any excess foil around the hermetically sealed end of container 2 then a pulse of very short duration may be detected. Again the microprocessor can be programmed with a range of acceptability criteria to ignore such short pulses, so code region 22 can be read successfully.

FIG. 9 is a flow diagram outlining the process of interrogating code region 22 of FIG. 5 by optical sensors 50, 52 and 54 within meter housing 32. Firstly, optical sensors 50, 52 and 54 are turned on by the microprocessor which itself is powered on by activation of a switch 44 by protrusion 12 on container 2 when inserted within strip port bay 42, step 250. As container 2 is withdrawn from strip port bay 42, step 252, clock hue sensor 50 reads clock line 84 looking for the first transition in voltage, step 254, i.e. either a tail below 1.3 V (transition from a dark patch 82 to a light patch 80) or a rise above 1.7V (transition from a light patch 80 to a dark patch 82) as discussed in relation to FIGS. 7 and 8. A timer may alternatively be activated on detection of the first clock, edge, step 260. For each clock edge detected, step 256, a count of clock edges stored by the microprocessor increases by 1, step 262, and first and second data line sensors (54 and 52) are triggered to sample first and second data lines (86 and 88) respectively at that point in time, step 264. As container 2 continues to be withdrawn bum strip port bay 42 the next clock edge in clock line 84 is detected by clock line sensor 50 and so on until all clock edges present are detected and corresponding data line information read by data line sensors 52 and 54.

When no further clock edges are detected at step 256, the count of clock edges is compared against the number of clock edges expected to have been detected, step 266. If the numbers match, all data bus read by data line sensors 52 and 54 are decoded by software within the microprocessor, step 268, and the corresponding calibration code stored within the memory of the meter for subsequent use in the final calculation of an analyte concentration. As mentioned previously, feedback may alternatively he provided to the user via an audible beep for example or a message displayed on display 36, and alternatively the user may be requested to verify that the calibration code was successfully retrieved, and is correct, step 270. All optical sensors 50, 52 and 54 are powered down immediately after retrieval of the optical coded data, step 272, and the user may proceed with the test, step 274.

If, however, the number of clock edges counted does not match the number of clock edges expected at step 266 i.e. the number of clock edges programmed within the meter software, then an error message may be displayed to the user informing them that the coded information was not read successfully, step 276. If an error occurs, for example if container 2 was withdrawn from strip port bay 42 either too quickly or too slowly or if it was moved in such a way that prevented successful reading of code region 22, then the user may alternatively be requested to re-insert container 2 in a further attempt to read the coded information successfully, step 278. If a user only partially removes container 2, re-inserts it slightly then continues to remove it completely, switch 44 may again be triggered by protrusion 12. If this were to happen, then reading of code region 22 would be re-set and read again as container 2 was removed successfully. Re-setting of the code reading procedure ensures that the correct calibration information is read.

In one embodiment, the calibration code can be read twice and the data from both readings are compared to determine if they are the same to ensure a correct reading. If there is a difference with both data then the test meter can output a signal (e.g., sound or display) indicative of any difference in the data read during the removing of the test strip and data read during the inserting of the test strip.

Alternatively, the user may be provided with the ability to enter the calibration code, step 280, allowing them to continue with the test, step 274, knowing that the meter is calibrated for the specific test strip being used.

A timer may be activated when the first clock edge is detected by clock line sensor 50, step 258, in order for the overall time taken to completely withdraw container 2 from strip port bay 42 to be measured. Each user will withdraw container 2 at slightly different speeds and optical sensors 50, 52 and 54 are required to interrogate code region 22 successfully whether container 2 is withdrawn relatively quickly, as shown in FIG. 7 or relatively slowly as shown in FIG. 8. Determining the timing or frequency of detection of clock edges enables the meter to calculate the speed of removal of container 2 from strip port bay 42, and thereby determine a maximum speed at which container 2 may be withdrawal aiding in the detection of any errors associated with the dynamic, optical reading of code region 22.

Alternatively, the calibration code may be provided on container 2 with the optical reader housed within a meter as described herein. Alternatively, the calibration code of the present invention may be now sled on individual test strips, with the optical reader housed within a meter. Yet in an alternative embodiment, the calibration code may be located, on a cassette or cartridge containing a plurality of test strips, and the optical reader again located within a cooperating meter. Alternatively the calibration code of the present invention may be located on a vial or other container storing one or more test strips, and may be used separately or in conjunction with a cooperating meter.

FIG. 10a is a further exemplary embodiment of an optical code pattern for use in the transfer of information, including a clock line 300 located in the center of code region 22 on top surface 4 of container 2, a first data, line 86 and a second, data line 88. In this exemplary embodiment, clock line 300 reads dark, light, dark, light, dark, light, dark resulting in 6 distinct clock edges 302, 304, 306, 308, 310 and 312 as defined in relation to FIGS. 5, 7 and 8.

FIG. 10b is a further still exemplary embodiment of an optical code pattern for use in the transfer of information, including the same features as the embodiment shown in FIG. 10a, however in this further example, clock line 400 reads light, dark, light, dark, light, dark resulting in 5 distinct clock edges 402, 404, 406, 408, 410 and 412.

After the clock hue data has beers read by clock line sensor 50, hut prior to the optical code being decoded, the meter software compares the number of clock edges detected against the number expected (step 266 in FIG. 9) and hence checks the pattern of data bits present. Referring now to FIGS. 10a and 10b, a clock line within code region 22 on individual containers 2 can be different for example code region 22 in FIG. 10a shows 6 clock edges whereas code region 22 in FIG. 10b shows only 5 clock edges, which may be altered specific to the expected market of the batch of containers (containing a test strip, optional with integrated lancet). For example, meters and containers for use in a particular country may be programmed with the relevant language for that country, and may also automatically trigger a welcome screen in the correct language for the expected recipients. Varying the bit pattern of clock line 300, 400 can provide users with the added advantage of recognized language defaults, user interlace defaults, a welcome greeting in the correct language and potentially provision of the correct customer services number should further advice be required by the user.

FIG. 11 is a cross-section view through strip port bay 42 of meter 80 of FIG. 1B showing the relative locations of calibration optical sensors 50, 52 and 54 and a strip presence detection sensor 56 according to a further embodiment of the present invention, including a front side 42a of strip port bay 42, a back side 42b of strip port bay 42, a test strip 500, a lancet portion 502 and a strip port connector 504. Test strip 500 including an integrated lancet portion 502 in one embodiment, is securely held within SPC 504 for the duration of the test measurement, and is both loaded into meter 30 and removed by means of container 2. Use of container 2 facilitates easy handling of small test strips, and more specifically prevents direct handling of used strips contaminated with a sample such as blood. Following a test, the user re-inserts container 2 into snip port bay 42 in order to dislodge the used test strip 500 from SPC 504, and re-engage the test strip 500 with engaging features within container 2. Container 2 is then safely disposed of, reducing the possibility of another person coming into contact with a sharp and contaminated test strip 500.

It is preferred that the test strip 500 properly engages within SPC 504 to ensure meter 30 provides a reliable measurement. Incorrect loading of a test strip 500 into SPC 504 may result in an error message being displayed to the user, perhaps requesting that container 2 be re-inserted to attempt to correctly engage strip 500 with SPC 504. If meter 30 had no ability to detect whether a strip 500 was correctly positioned or not, then it is believed that an incorrect result could be generated, or test strips 500 may potentially be wasted if the user has to re-test using another strip 500. It is therefore a further embodiment to provide a strip detection sensor 56 located within strip port bay 42 to detect and subsequently communicate to the microprocessor that a test strip 500 has been successfully loaded into meter 30.

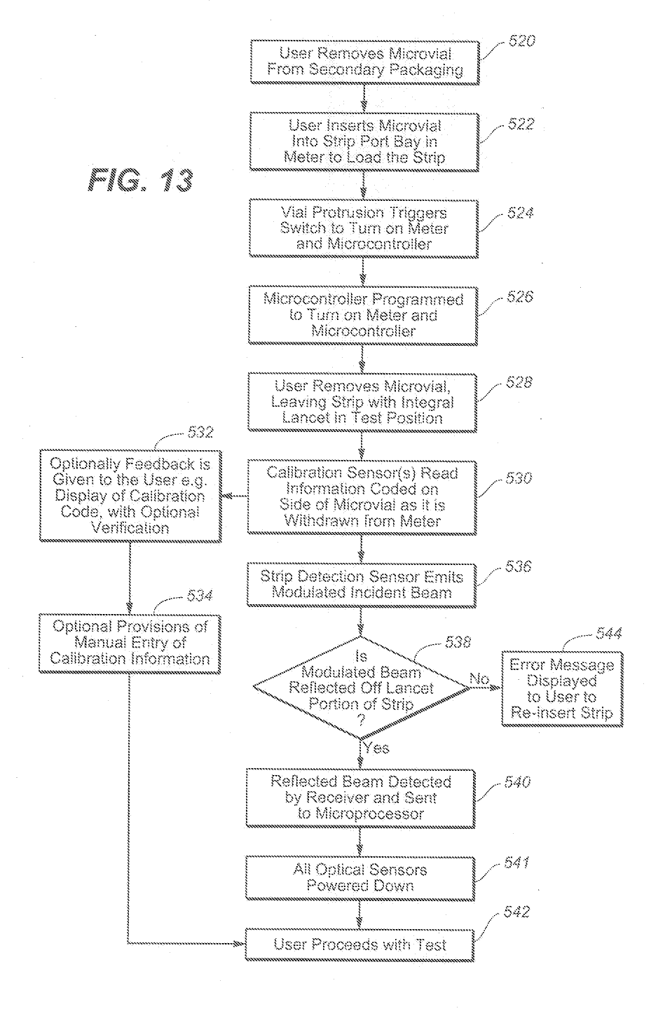

Strip port bay 42 may therefore include both an optical sensing system designed to interrogate a calibration code printed on one side of container 2 (sensors 50, 52 and 54), and also a strip detection sensor 56. Code sensors 50, 52 and 54 may alternatively be located towards the entry to strip port bay 42 to enable accurate reading of code region 22 as container 2 is withdrawn from strip port bay 42. Strip detection sensor 56 is located, in one embodiment, in line with lancet portion 502 of test strip 500 as detection sensor 56 operates by detecting light reflected off lancet portion 502, as will be described in more detail in relation to FIG. 13.

FIG. 12 is a schematic view of a test sensor 500 with an integrated lancet portion 502 for use with container 2 of FIGS. 1, 2 and 3, including electrodes 500 a microcontroller 508, a front end circuitry 510, a strip detection optical sensor 56 comprising an emitter portion 56a and a receiving component 56b, a direction of an incident beam of light denoted by arrow ‘H’ and a direction of a reflected beam of light denoted, by arrow ‘I’.

Strip detection sensor 56 operates by use of an emitter LED portion 56a and a receiving portion 56b positioned on backside 42b of strip port bay 42, in careful alignment with lancet portion 502 of strip 500. Emitting LED portion 56a sends a modulated light beam in a direction indicated by arrow ‘H’, which is reflected off lancet portion 502, indicated by arrow ‘I’, when a test strip 500 is correctly loaded into the SPC 504 component of meter 30. The reflected light ‘I’ is detected by receiving portion 56b, and the information received is sent via front-end circuitry 510 to a microcontroller 508. Microcontroller 508 demodulates the signal received from receiving portion 56b, and determines whether a test strip is correctly positioned in SPC 504.

Incorporating a strip detection sensor 56 into strip port bay 42, reduces the complexity of the strip port connector 504. Commercially available strips, such as the OneTouch® Ultra brand, from Lifescan. Inc., Milpitas, Calif., USA, include an additional bar printed on the end of the test sensor that engages with the SPC to instruct the meter to turn on. Removing toe need for this switch-on bar enables test strips to be designed smaller, thereby increasing manufacturing throughput.

FIG. 13 is a flow diagram outlining the main steps revolved in the procedure of loading a new test strip 500 into the meter 30 of FIG. 1B, which includes many of the same steps described in relation to FIG. 4. First the user removes container 2 from any form of secondary packaging, step 520. Container 2 is then inserted into strip port bay 42 to load a test strip 500 into meter 30 ready to perform a test, step 522. Once the user detects a prominent ‘click’ then container 2 is inserted far enough for test sensor 500 to engage with SPC 504, step 524. Container 2 triggers a switch 44, step 524, within strip port bay 42 that activates the calibration and strip detection optical sensors, step 526. On feeling the click, the container may be removed by the user, leaving a strip 500 loaded in meter 30, step 528. During withdrawal of container 2 optical code region 22 is read by optical sensors 50, 52 and 54, step 530. Strip detection sensor 56 subsequently detects whether or not a strip 500 is successfully loaded into meter 30 by emitting a modulated beam of light towards the lancet portion 502 of test strip 500, step 536, and detecting and demodulating the signal that is returned, step 538. Optical sensors 50, 52, 54 and 56 are subsequently powered down immediately after retrieval of information, step 541. If no strip 500 is present, an error message may be displayed to the user, step 544, requesting them to re-insert the strip. If the emitted beam of light is reflected off lancet portion 502 of test strip 500, then receiver 56b detects this signal and the information is sent to microcontroller 508, step 540. The user may then begin to test, step 542,

Alternatively, feedback, may be provided to the user, step 532, e.g. in the form of an audible beep or a message briefly displayed on display 36, and the user may alternatively be requested to verify the calibration information obtained from optical code region 22, or alternatively enter the calibration information manually, step 534. The user may then proceed with, the measurement procedure step 542, knowing that meter 30 is calibrated for the particular batch of test strips being used.

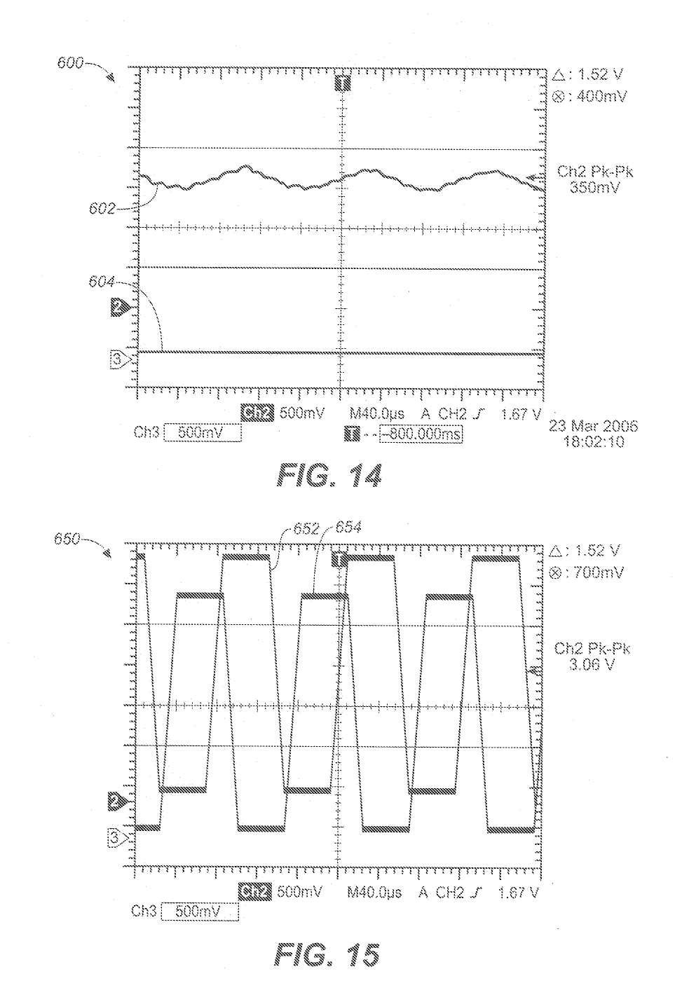

FIG. 14 illustrates an exemplary oscilloscope wave chart 600 obtained if a test ship 500 including integral lancet portion 502 is absent or incorrectly loaded into strip port connector 504, including an un-buffered signal 602 and a buffered signal 604.

FIG. 15 is an oscilloscope wave chart 650 of the optical detection of lancet portion 502 of test strip 500 of FIG. 12, detected once container 2 is withdrawn from strip port bay 42 of meter 30, including an un-buffered signal 652 and a buffered signal 654.

Referring now to FIGS. 14 and 15, beam of tight ‘H’ emitted from emitting portion 56a of strip detection sensor 56 towards lancet portion 502 of test strip 500, is reflected and the returned signal ‘I’ detected by receiving portion 56b and the information transferred to microcontroller 508. If a test strip 500 is successfully loaded into strip port bay 42 of meter 30, then a sinusoidal signal such as buffered signal 654 is sent to microcontroller 508, corresponding to a reflection of incident beam ‘H’ emitted, by emitting portion 56a, indicating that a test strip 500 is present and the user may proceed to test. If no test strip were loaded in strip port bay 42 of meter 30, then a flat signal such as buffered signal 604 would be received by microcontroller 508. Should a user fail to successfully load a test sensor 500 into meter 30, then an error message may be displayed requesting the user to re-insert the strip 500 as described in relation to FIG. 13.

Use of a modulated signal also overcomes any interference associated with sunlight entering into strip port hay 42. Meter 30 is therefore able to work reliably in all levels of sunlight experienced in different countries. Communication of strip detection sensor 56 with microcontroller 208 provides information on the presence or absence of a test strip 500 and allows the meter 30 to act accordingly i.e. provision of an error message to the user, or a request to re-insert container 2 to properly engage ship 500 with SPC 504. Such an optical detection system also provides real-time feedback to the user regarding the reliability of their measurement system.

Various embodiments described herein may provide many advantages, including removing the need for the user to input the calibration code, thereby reducing the number of user steps required for a user to perform a test. Calibration of an analyte monitoring meter, such as the example provided herein may be completely invisible to the user, providing them with a reliable system correctly calibrated irrespective of which batch-specific calibration code is assigned to the test strips being used.

A further advantage is provided by the fact that the optical sensors are only powered on for a short period of time, approximately 1 to 2 seconds, thereby reducing power consumption and hence eliminating the need for a large, expensive battery. Triggering the optical sensors to power on only when a container 2 is inserted into meter 30 prevents inefficient use of battery power, and the possibility of the optical sensors turning on accidentally is virtually eliminated as activation switch 44 (that activates microcontroller 508 that in turn powers on optical sensors 50, 52, 54 and 56) is protected within stop port bay 42.

Another advantage is the technique of reading the data by movement of the container (i.e., dynamic code readings rather than scanning movement by the optical reader against a stationary container, thereby obviating the need for a complex scanning mechanism to scan the data.

Yet another advantage results from the use of dynamic reading is the utilization of one optical sensor per data line of calibration information. This is believed to provide advantages over static code reading methods where one optical sensor is required per individual bit of information. That is for a 10-bit device, 10 optical sensors may be needed, potentially resulting in a large, more costly measurement device.

A further advantage is the use of an optical strip detection system in cooperation with the optical calibration code sensors. Use of optical sensors allows the strip port connector to be smaller and less complex, and also allows smaller test strips to be manufactured, as no switch-on bar is required. Small test strips are desirable in measurement systems where the user does not have to handle the strips directly, such as the container method described herein. Whilst the use of both a calibration code sensor(s) and a strip detection sensor is discussed herein, it would be apparent to a person skilled in the art that the sensors discussed may each be used alone or in combination.

By virtue of the above description provided herein, various methods of transmitting data specific to a test media to a medical test device can be achieved. For example, one preferred method may involve inserting the container into a test strip receptacle or port of the test device; removing the container out of the test port; and reading the discrete surface features as the container is moving relative to the test port during one of the removing and inserting steps to provide data specific to the test strip. In one particular embodiment, the reading includes recognizing the surface features during the inserting. In another particular embodiment, the reading includes recognizing the surface features during the removing. In yet another embodiment, the reading includes decoding data encoded by the surface features during the inserting and removing; comparing data during the removing with data during the inserting; and outputting a signal such as, for example, sound or visual display to reflect any difference in the data read during the removing and data, read during the inserting. It is also preferred that the test strip is retained to the sampling port upon removal of the container. It is noted that in reading the discrete surface features, there is recognition of the transitions between discrete features of the second plurality of discrete surface features of the clock line. Further, the method involves correlating the transitions of the clock line to transitions between the first plurality of discrete surface features, and providing binary data from the correlating.

The method may involve confirming that the at least one test strip is retained in the port; and reading the discrete surface features during one of the removing and inserting and only upon confirmation by the confirming step. The reading of the data may involve verifying such reading with a human observable, output by the test device. Finally, the method may include validating the binary data that were read with prestored binary data in the test device to ensure that the test strip is an authentic test strip.

While the invention has been described in terms of particular variations and illustrative figures, those of ordinary skill in the art will, recognize that the invention is not limited to the variations or figures described. For example, more than one strip can be utilized in a container where the strips are made in a batch having specific calibration parameters. In addition, where methods and steps described above indicate certain events occurring in certain order, those of ordinary skill in the art will recognize that the ordering of certain steps may be modified and that such modifications are in accordance with the variations of the invention. Additionally, certain of the steps may be performed concurrently in a parallel process when possible, as well as performed sequentially as described above. Therefore, to the extent there are variations of the invention, which are within the spirit of the disclosure or equivalent to the inventions found in the claims, it is the intent that this patent will cover those variations as well. Accordingly, it is intended that the present invention not be limited to the described embodiments, but that it have the full scope defined by the language of the billowing claims, and equivalents thereof.

What is claimed is:

1. A container comprising:
an interior surface defining an internal volume;
a test strip disposed in the internal volume, the single test strip having at least a calibration code corresponding to predetermined calibration parameters specific to the single test strip;
an outer surface that surrounds a substantial portion of the interior surface;
a first plurality of discrete surface features disposed on the outer sort see to define a first data line; and
a second plurality of discrete surface features disposed on the outer surface in a repeating sequence to define timing intervals for the first data line so that the calibration code of the single test strip is encoded by the first and second plurality of discrete surface features.

2. The container of claim 1, further comprising a third plurality of discrete surface features disposed on the outer surface to define a second data line.

3. The container of claim 2, wherein the first and second data lines comprise at least 4 data bits of information.

4. The container of claim 3, wherein the first and second data lines comprise at least 10 data bits of information indicative of the calibration code.

5. The container of claim 4, wherein the data lines further comprise information selected from a group consisting essentially of geographical data, error checking data, lot number, manufacturing date, expiration date, batch number, manufacturer's name, chemical composition, ingredients, and combinations thereof

6. The container of claim 2, wherein each of the first, second and third plurality of discrete surface features comprises a plurality of low and high reflectance areas.

7. The container of claim 2, wherein each of the first, second and third plurality of discrete features comprises a plurality of raised and depressed surfaces formed on the outer surface.

8. The container of claim 4, wherein the second plurality discrete surface features comprise a clock line contiguous the two data lines, the clock line configured to indicate that the stop is for distribution to a user in a predetermined geographic location.

7. The container of claim 6, wherein the clock line comprises a series of generally identical polygons disposed in a generally linear sequence between the first and second data lines.

8. The container of claim 6, wherein each of the low and high reflectance areas comprises respective ink materials with discrete reflectance values.

9. The container of claim 8, wherein each of the ink materials is configured to degrade over a predetermined duration.

10. The container of claim 5, wherein the test strip comprises a lancet affixed to a glucose test strip.

11. The container of claim 6, wherein the clock and data lines comprise respective parallel arrays of discrete reflectance areas.

12. A medical analyte measurement system comprising:
a medical test media container including:
an interior surface defining an internal volume;
a test step disposed in die internal volume, the test strip having a calibration code corresponding to predetermined calibration parameters of the test strip; and
an outer surface that surrounds a substantial portion of the interior surface, the outer surface having discrete surface features indicative of the calibration code of the test strip; and
a medical test device including;
a sample port configured to receive the lest strip container in only one spatial orientation; and
a pattern reader configured to recognize at least the calibration code encoded in the discrete surface features upon relative movement between the container and fire sample port,

13. The system of claim 12, wherein the pattern reader recognizes the discrete surface features without physical contact between the pattern reader and the discrete surface features.

14. The system of claim 12, wherein the discrete surface features comprise:
a first plurality of discrete surface features disposed on the outer surface to define a first data line; and
a second plurality of discrete surface features disposed on the outer surface in a repeating sequence along a second perimeter to define timing intervals for the first data line.

15. The system of claim 12, wherein each of the first and second plurality of discrete surface features comprises a plurality of raised and depressed surfaces formed on the outer surface.

16. The system of claim 14, wherein the first plurality of discrete surface features includes a first plurality of low and high reflectance areas disposed on the outer surface to define a first data line; and the second plurality of discrete surface features includes a second plurality of low and high reflectance areas disposed on the outer surface in a repeating sequence along a second perimeter to define timing intervals for the first data line.

17. The system of claim 13, wherein the pattern reader comprises a plurality of optical detectors configured to read the discrete surface features and presence of the test strip in the sample port.

18. The system of claim 14, wherein the first and second data lines comprise at least 4 data bits of information.

19. The system of claim 14, wherein the pattern reader comprises three optical sensors and the first and second data lines include 10 data bits.

20. The system of claim 19, wherein the data lines hinder comprise information selected from a group consisting essentially of geographical data, error checking data, lot number, manufacturing date, expiration date, batch number, manufacturer's name, chemical composition, ingredients, and combinations thereof.
